(19) United States
(12) Patent Application Publication
(10) Pub. No.: US 2026/0224200 A1
(43) Pub. Date: Aug. 6, 2026

(54) PROBE POSITIONING DEVICE HAVING GAS AND LIQUID REMOVAL MECHANISM

(71) Applicant: CIVCO MEDICAL INSTRUMENTS CO., INC., Kalona, IA (US)

(72) Inventor: Willet Whitmore, III, Longboat Key, FL (US)

(21) Appl. No.: 19/148,304

(22) PCT Filed: Jan. 11, 2024
(86) PCT No.: PCT/US2024/011170

§ 371 Date: Jul. 15, 2025

Related U.S. Application Data
(60) Provisional application No. 63/480,410, filed on Jan. 18, 2023.

(51) Int. Cl. A61B 8/00; A61M 25/02; A61M 25/10

(52) U.S. Cl. CPC A61B 8/445; A61M 25/10; A61M 2025/0246

(57) ABSTRACT

An apparatus comprises a flexible sheath body configured to receive at least a portion of an ultrasound probe having a transducer portion. A balloon portion is secured to the sheath body proximate or adjacent to the transducer portion, wherein the balloon portion includes a fluid introduction port for allowing fluid to inflate the balloon portion. A gas and liquid removal portion comprises gas and liquid removal tubing having a plurality of gas and liquid removing holes therein, and a valve coupled to an open end of the gas removal tubing. The valve is configured to permit application of a source of negative pressure therein to the gas and liquid removal tubing to remove gases or liquids from within the gas and liquid removal tubing and from within a body cavity in which the transducer portion is placed.

### BACKGROUND

This invention relates to medical devices and more particularly to ultrasound probes and devices for positioning the same within a cavity or lumen in the body of a patient for enabling optimal imaging during performance of a transperineal (TP) prostate brachytherapy and some types of prostate biopsy.

TP prostate brachytherapy and some biopsy procedures generally require a patient to be placed in the dorsal lithotomy position rather than a lateral position, so that the perineal skin can be well and symmetrically exposed during the procedure. Unfortunately, such on-the-back patient positioning increases the likelihood that bowel gases or other fluids may become a significant problem for maintaining a good acoustic window for imaging.

Currently, when prostate gases or fluids are identified, the only solution is to remove the transducer from the patient's rectum, remove of the rectal gas and/or fluids with a suction catheter, and then reposition the transducer within the patient's rectum. However, transducer positioning is an important and somewhat exacting part of the TP prostate brachytherapy or biopsy procedure. When it is interrupted, it can be quite troublesome for multiple reasons including interference with the critical accuracy of radioactive seed placement. Accordingly, there remains a need for a device that supports prostate gas or fluid removal without requiring removal of a precisely positioned ultrasound probe.

### BRIEF DESCRIPTION OF THE DRAWING

FIG. 1 is a side plan schematic view of a probe positioning device in a first, uninflated state, consistent with embodiments described herein;

FIG. 2 is a side plan schematic view of the probe positioning device of FIG. 1 in a second, inflated state;

FIG. 3 is a cross-sectional view of the probe positioning device of FIG. 2 taken along the line A-A in FIG. 2;

FIG. 4 is a side plan schematic view of another probe positioning device in a first, uninflated state, consistent with embodiments described herein;

FIG. 5 is a side plan schematic view of the probe positioning device of FIG. 4 in a second, inflated state; and

FIG. 6 is cross-sectional view of the probe positioning device of FIG. 5 taken along the line B-B in FIG. 5.

### DETAILED DESCRIPTION OF THE PREFERRED EMBODIMENT

The following detailed description refers to the accompanying drawings. The same reference numbers in different drawings may identify the same or similar elements. Also, the following detailed description does not limit the invention.

Several embodiments of a probe positioning device and system are described that provide for removal of prostate gases and/or liquids during ultrasound imaging of a cavity e.g., a rectum, of a patient. Consistent with implementations described herein, such a probe positioning device may include a flexible sheath for surrounding the end of an ultrasound probe. The flexible sheath includes at least one inflatable region and a gas and liquid removal portion having a least one opening therein that may be exposed to any gas and liquid that may be present within the rectum. During use, fluid may be inserted into the inflatable region to provide an optimal acoustic imaging window. Additionally, in the presence of rectal gases or liquids that may be interfering with optimal ultrasonic imaging, a source of negative pressure (e.g., a syringe, a suction source, etc.) may be applied to the gas and liquid removal portion, to remove interfering rectal gases and/or liquids from the patient's rectum.

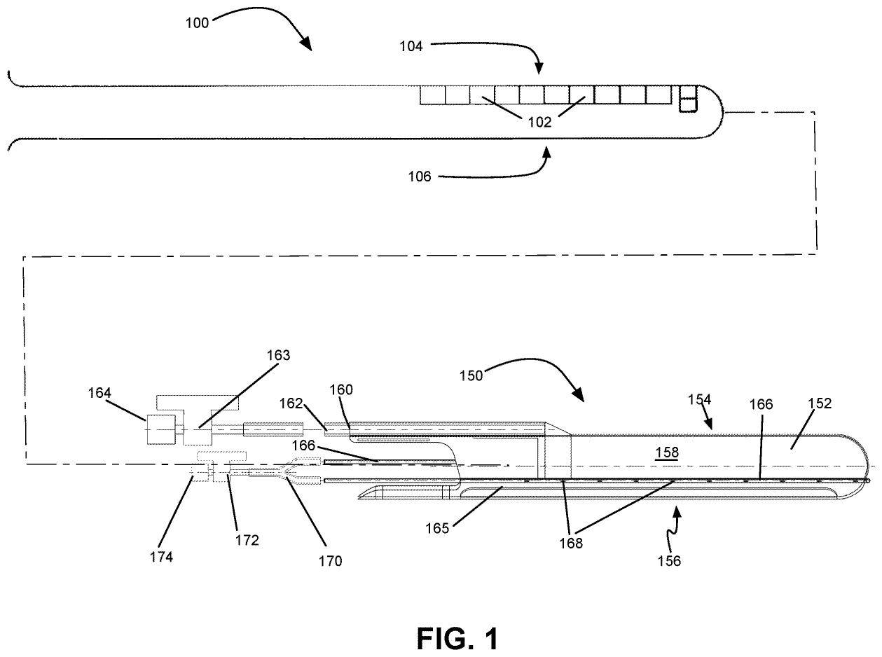

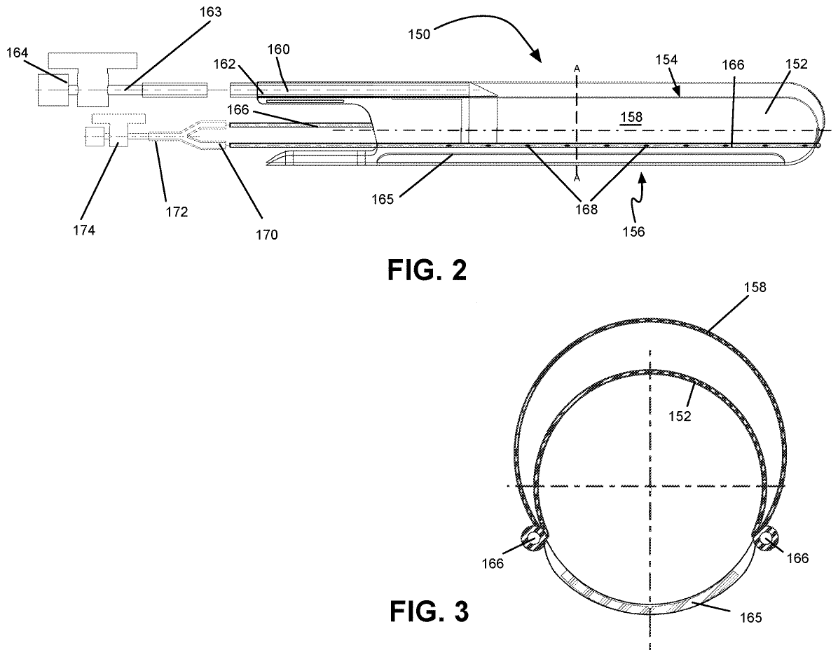

FIG. 1 is a side plan schematic view of a probe positioning device 150 depicted in relation to an ultrasound probe 100 in an initial or uninflated state consistent with implementations described herein. FIG. 2 is a side plan schematic view of probe positioning device 150 in a second or inflated state consistent with implementations described herein. FIG. 3 is a cross-sectional end view of the probe positioning device 150 of FIG. 2 taken along the line A-A in FIG. 2.

As shown in FIG. 1, ultrasound probe 100 has a generally cylindrical body and includes one or more ultrasound transducers 102 positioned in a transducer region 104 of probe 100 that transmit and receive ultrasound waves for generating images of tissues. In some implementations, the diameter of the probe 100 is approximately 2 cm, and the length of the transducer array can be up to 10 cm. Probe 100 further includes an underside region 106 located at the diametrically opposite side from the transducer region 104.

As shown in FIGS. 1 and 2, probe positioning device 150 includes a sheath body 152, an inflatable portion 154, and a gas removal portion 156. Sheath body 152 comprises a snugly fitting, condom-like member formed of a resilient flexible material, e.g., latex, polyurethane or any other suitable material.

Inflatable portion 154 includes a balloon portion 158 affixed (e.g., glued, welded, etc.) to a portion of sheath body 152 proximate to transducer region 104 of probe 100 during use. Inflatable portion 154 includes an opening 160 that forms a fluid introduction port coupled to a fluid introduction tube 162. Fluid introduction tube 162 is coupled to a manually operated valve 163, such as a stopcock, which is then coupled to a fitting 164 for receiving a fluid therethrough.

In some implementations, balloon portion 158 may be formed of a material that is more elastic material than that of sheath body 152. However, in an alternative embodiment balloon portion 158 may be identical to the material of sheath body 152.

As shown in FIGS. 1-3, gas removal portion 156 may include a batten pocket 165, and gas removal tubing 166. Batten pocket 165 is affixed (e.g., glued, welded, etc.) to a portion of sheath body 152 proximate to underside region 106 of probe 100 during use. As shown in FIGS. 1-3, batten pocket 165 may be open at its rear end to allow access to sheath body 152 during use. As with balloon portion 158, batten pocket 165 may also be formed of a different or same material as that of sheath body 152.

As shown in FIGS. 1 and 2, gas removal tubing 166 may include a U-shaped length of tubing secured (e.g., glued) to sheath body 152 between balloon portion 158 and batten pocket 165 so as to effectively wrap longitudinally around the tip of ultrasound probe 100 during use. Consistent with implementations described herein gas removal tubing 166 may be formed of ⅛″ outside diameter polyurethane tubing although other sizes or materials may be used. As shown, gas removal tubing may include a plurality of gas aspiration holes 168 formed in a periphery thereof in a portion of gas removal tubing 166 proximate to balloon portion 158.

In one implementation, gas removal portion 156 further comprises a Y-connector 170 for joining the open ends of gas removal tubing 166. Y-connector 170 is coupled to a manually operated valve 172, such as a stopcock, which is then coupled to a fitting 174 for receiving a source of negative pressure.

During use, ultrasound probe 100 is inserted into probe positioning device 150 and the combination is inserted into a patient's rectum. Fluid is administered into balloon portion 158 via fluid introduction tube 162 to inflate balloon portion 158 and position ultrasound probe 100 within the rectum. If or when image compromising prostate gases and/or liquids are identified, an operator may attach a source of negative pressure, such as a syringe, to fitting 174 of gas removal portion 156 and may open valve 172 to allow prostate gases and/or liquids present in the rectum to be removed via the removal tubing 166. Consistent with the embodiment of FIGS. 1-3, the combination of ultrasound probe 100 and probe positioning device 150 may need to be rotated plus or minus 90° clockwise/counterclockwise about its axis for the device to function optimally for gas aspiration.

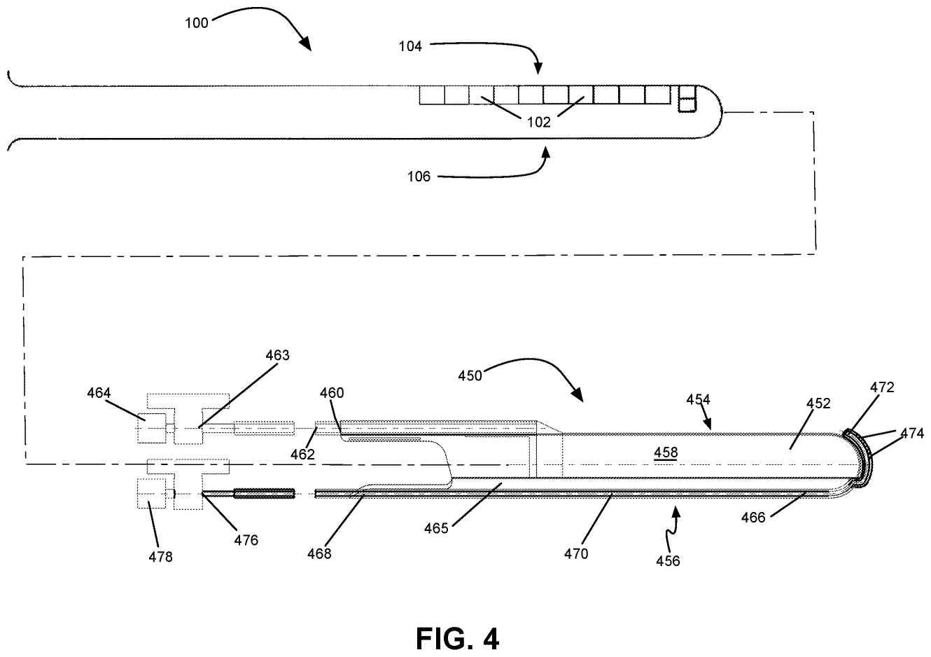

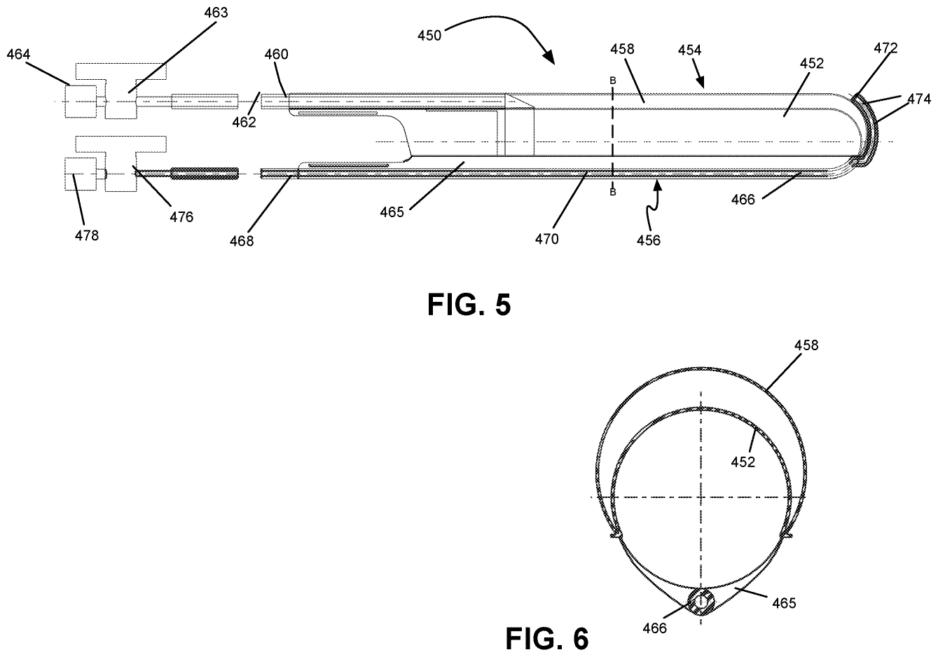

FIG. 4 is a side plan schematic view of a second embodiment of a probe positioning device 450 depicted in relation to ultrasound probe 100 in an initial or uninflated state consistent with implementations described herein. FIG. 5 is a side plan schematic view of probe positioning device 450 in a second or inflated state consistent with implementations described herein. FIG. 6 is a cross-sectional end view of the probe positioning device 450 of FIG. 5 taken along the line B-B in FIG. 5.

As shown in FIGS. 4 and 5, probe positioning device 450 includes a sheath body 12, an inflatable portion 454, and a gas removal portion 456. Sheath body 452 comprises a snugly fitting, condom-like member formed of a resilient flexible material, e.g., latex, polyurethane or any other suitable material.

Inflatable portion 454 includes a balloon portion 458 affixed (e.g., glued, welded, etc.) to a portion of sheath 454 proximate to transducer region 104 of probe 100 during use. Inflatable portion 454 includes an opening 460 that forms a fluid introduction port coupled to a fluid introduction tube 462. Fluid introduction tube 462 is coupled to a manually operated valve 463, such as a stopcock, which is then coupled to a fitting 464 for receiving a fluid therethrough. Similar to balloon portion 158 described above, in some implementations, balloon portion 458 may be formed of a material that is more elastic material than that of sheath body 452.

As shown in FIGS. 4-6, gas and/or liquid removal portion 456 may include a batten pocket 465, and gas/liquid removal tubing 466. Batten pocket 465 is affixed (e.g., glued, welded, etc.) to a portion of sheath 454 proximate to underside region 106 of probe 100 during use. As shown in FIG. 6, batten pocket 465 may be open at its rear end to allow access to sheath body 452 during use. As with balloon portion 458, batten pocket 465 may also be formed of a different or same material as that of sheath body 452.

As shown in FIGS. 4 and 5, gas removal tubing 466 may include length of tubing having a proximal portion 468, an intermediate portion 470, and a distal end 472. As shown, intermediate portion 470 of gas/liquid removal tubing 466 extends through batten pocket 465. Distal end 472 is secured (e.g., glued) to a distal end 459 of balloon portion 458. As shown in FIG. 5, such a configuration causes gas removal tubing 466 to curve substantially perpendicularly between intermediate portion 470 and distal end 472.

As shown in FIGS. 4 and 5, distal end 472 of gas/liquid removal tubing 466 includes a plurality gas aspiration holes 474 formed therein. In one implementation, proximal portion 468 of gas/liquid removal tubing 466 is coupled to a manually operated valve 476, such as a stopcock, which is then coupled to a fitting 478 for receiving a source of negative pressure. Consistent with implementations described herein gas/liquid removal tubing 466 may be formed of ⅛″ outside diameter polyurethane tubing although other sizes or materials may be used.

During use, ultrasound probe 100 is inserted into probe positioning device 450 and the combination is inserted into a patient's rectum. Fluid is administered into balloon portion 458 via fluid introduction tube 462 to inflate balloon portion 458 and position ultrasound probe 100 within the rectum. Because gas/liquid removal tubing 466 is not secured to batten pocket 465, but only to distal end 459 of balloon portion 458, as balloon portion 458 is inflated, intermediate portion 470 of gas/liquid removal tubing 466 is permitted to slide within batten pocket 465.

If or when interfering rectal gases or liquids are identified, an operator may attach a source of negative pressure, such as a syringe, to fitting 478 of gas/liquid removal portion 456 and may open valve 476 to allow the interfering rectal gases and/or liquids present into the gas/liquid removal tubing 466 to be removed.

The foregoing description of exemplary implementations provides illustration and description but is not intended to be exhaustive or to limit the embodiments described herein to the precise form disclosed. Modifications and variations are possible in light of the above teachings or may be acquired from practice of the embodiments.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements. Similarly, relative terms, such as “upper/lower”, “front/rear”, and “forward/backward” are used to depict relative positioning with respect to described components and do not refer to absolute or gravity-based relative positions. Embodiments described herein may be implemented in any suitable orientation.

What is claimed is:

1. An apparatus comprising:
a flexible sheath body configured to receive at least a portion of an ultrasound probe having a transducer portion;
a balloon portion secured to the sheath body proximate the transducer portion,
wherein the balloon portion includes a fluid introduction port for allowing fluid to inflate the balloon portion; and
a gas and liquid removal portion comprising:
gas and liquid removal tubing having a plurality of gas and liquid removing holes therein, and
a valve coupled to an open end of the gas and liquid removal tubing,
wherein the valve is configured to permit application of a source of negative pressure therein to the gas and liquid removal tubing to remove gases or liquids from within the gas and liquid removal tubing and from within a body cavity in which the transducer portion is placed.

2. The apparatus of claim 1, further comprising a batten pocket secured to the flexible sheath body at a position opposite to the balloon portion.

3. The apparatus of claim 2, wherein the gas and liquid removal tubing passes through the batten pocket and is secured to a distal end of the balloon portion.

4. The apparatus of claim 3, wherein the plurality of gas and liquid removing holes are formed in a distal end of the gas and liquid removal tubing proximate to the balloon portion.

5. The apparatus of claim 1, wherein the gas and liquid removal tubing comprises U-shaped tubing that wraps longitudinally around the sheath body, wherein the U-shaped tubing is secured to a periphery of the sheath body.

6. The apparatus of claim 5, wherein the plurality of gas and liquid removing holes are formed in portions of the gas and liquid removal tubing proximate or adjacent to the balloon portion.

7. The apparatus of claim 5, further comprising a Y-connector for joining open ends of the U-shaped tubing,
wherein the valve is coupled to the Y-connector.

8. The apparatus of claim 1, wherein the gas and liquid removal tubing comprises ⅛″ outside diameter polyurethane tubing.

9. The apparatus of claim 1, wherein the valve comprises a stopcock.

10. The apparatus of claim 1, wherein the source of negative pressure comprises a syringe.
